(19) United States
(12) Patent Application Publication
(10) Pub. No.: US 2006/0222705 A1
(43) Pub. Date: Oct. 5, 2006

(54) PHARMACEUTICAL TABLET AND APPARATUS AND METHOD FOR MAKING

(76) Inventors: Henry H. Flanner, Montgomery Village, MD (US); Ronald Jackson Casey, North Bethesda, MD (US)

Correspondence Address: CARELLA, BYRNE, BAIN, GILFILLAN, CECCHI,;STEWART & OLSTEIN, 5 BECKER FARM ROAD, ROSELAND, NJ, 07068, US

(21) Appl. No.: 11/277,831

(22) Filed: Mar. 29, 2006

Related U.S. Application Data
(60) Provisional application No. 60/666,972, filed on Mar. 31, 2005.
(60) Provisional application No. 60/775,838, filed on Feb. 22, 2006.

(51) Int. Cl. A61K 9/20

(52) U.S. Cl. 424/464

(57) ABSTRACT

A punch and die set include a die with a single large cavity which can receive dry particles of relatively large dimensions or large arching indexes as compared to the transverse dimensions of the tablets formed by the particles, e.g., 1.5 mm diameter and 1 mm height. The upper and lower punches are configured to form an array of interconnected tablets. The punch and dies compress the particles in the die cavity into the array. The tablets are interconnected by an array of links formed by tangential regions of adjacent cylindrical tablets or by links or bridges of finite length, width and thickness dimensions as defined by the punch configurations. The tablets of the arrays are separated by forces created by vibratory or tumbling action. Various embodiments of different shaped tablets and mating punch and die sets are disclosed. A tablet forming apparatus by way of example feeds medicament powder horizontally radially outwardly to the punch and dies by centrifugal force via a rotating powdered medicament receiving hopper.

This application claims the benefit of provisional applications Ser. No. 60/666,972 filed Mar. 31, 2005, and Ser. No. 60/775,838 filed Feb. 22, 2006, entitled “Pharmaceutical Tablet and Apparatus and Method For Making” incorporated by reference herein in their entireties.

This invention relates to miniature pharmaceutical tablets, e.g., about 1.5-2 mm or smaller in diameter, comprising dry granular powder or equivalent thereof, and apparatus and methodology for making such tablets.

Pharmaceutical tablets are formed of dry powder particles. Such pharmaceutical particles are irregular and heterogeneous in shape. In fabrication of capsules or sachets for sprinkle applications, small pellets or tablets, e.g., 1-2 mm or smaller in diameter, typically coated to permit time variation in the release of the medication, are used. See for example, U.S. Pat. No. 3,175,521 ('521) related to the fabrication of miniature tablets of 1/16 and ½ inch diameter, incorporated by reference herein.

Tablets are formed of dry particles and are formed by upper and lower mating punches which cooperate with an intermediate die having a cavity receiving the particles. The particles are compressed in the die by the punches. See the aforementioned '521 patent. The problem with such dies for use in making tablets of about 1.5-2 mm diameter or smaller, is that such dies do not readily and reliably fill properly due to the irregular shape of the particles, low density of the pharmaceutical powder material, and wide particle size distribution of the powders common in the pharmaceutical industry as well as by variation in their arching index. This results in unacceptable variation of weight and hence dosages, and resultant physical properties such as hardness and friabilility among the tablets.

For example, typical pharmaceutical powder blends that are required to flow into a die cavity in a punch tablet process are not uniform in size, shape or morphology. Some are longer than about 0.7 mm, some are shorter. This makes the flow into the die difficult, since orientation of powder particles to die cavity orientation is random, thus increasing the probability that larger particles will “bridge” or “arch” over a fixed opening width, such as a circle, and thus block flow into the die cavity. Small constant diameter openings rule out the use of many pharmaceutical powders because their arching index is larger than the die cavity opening.

It goes to the reason why manhole covers are round. There is no way for a round manhole cover to orient to fall through a smaller diameter round manhole. But a square or oval manhole cover can orient in some way to fall through corresponding square or oval or some other non-round shape while others will not enter the same opening. Pharmaceutical particles of irregular size and shape have the same problem when “bridging” occurs over a constant diameter.

This problem with irregular shaped particles and bridging potential is present for the '521 disclosed apparatus. Therefore, this apparatus is not believed to be able to reliably produce small tablets of about 1.5 mm to provide the desired dosage tolerances needed in practice without further processing of the particles to eliminate irregular shapes and thus reduce the “Arching Index” of the powder so it can flow into the die cavity. This is wasteful and expensive in that some particles that are otherwise acceptable will be rejected.

Other patents disclosing apparatus or tablets of small dimensions include U.S. Pat. Nos. 4,828,843; 3,473,490; 4,339,428; and 4,294,819. The '843 patent discloses cylindrical microtablets having a diameter and height of about 1.0 to 2.5 mm with the ratio of diameter to height from 1:0.5 to 1:1.5 and a process for their preparation. The particles are produced by milling and then pressed. The '428 patent discloses a capsule product and a tablet punch and mold. The '819 patent discloses a capsule containing aspirin and is made stable with alkaline material in tablet form. The '490 patent discloses passing the punch through a reservoir of a mass of powder and forcing the precompressed powder into a die cavity.

Pellets as defined herein are formed by a wet process. The wet process is an aqueous extrusion and spheronization process typically used to make pellets of less than 1.5 mm. A wet mass of powder is extruded into “spaghetti strands.” The strands are then spheronized to make approximately round particles (typically called pellets) of anywhere from 200 microns to 2 mm. These are then dried in an oven or in a fluidized bed dryer. Pellets formed by the extrusion and spheronization process have a particle size distribution that is dependent on the properties of the powder and the process parameters

A narrow distribution is desirable, but not always achievable by extrusion and spheronization. The approximately spherical shape makes the pellets good substrates for coating, but a wide distribution of sizes makes coating more difficult, and less uniform and smaller particles typically require more coating. Pellets are not tablets and are made by a completely different process, i.e., the wet extrusion and spheronization process described. Sizes of less than 1.5 mm are desirable for use in small gelatin capsules (a gelatin or other hollow digestible casing receiving the pellets) or pouch or sachet type products so they can be administered to pediatric and geriatric patients that have difficulty swallowing. The pellets and tablets typically are coated to provide an active medicine for use over a given time period. The small pellets and tablets are also useful by sprinkling over food stuff such as apple sauce and the like so they are readily swallowed by children and elderly persons.

Uniformity of dosage is important. When the dosage varies among the tablets, capsules containing such tablets exhibit wide variation of dosage values and performance, which is not acceptable In addition, when the weight of tablets varies, the physical properties of the tablets can change. Low weight tablets may not have been completely compressed and may fall apart or break apart on later handling, a symptom of low hardness or high friability. High weight tablets may be over compressed and suffer from slow disintegration or slow dissolution. Tablets of less than 1.5 mm are a rarity commercially regardless of the above noted patents due to the practicality of producing such tablets consistently in large numbers over time. Pellets made with a wet extrusion and spheronization or drug layering process and of less than 1.5 mm are common.

Tablets of about 1.5 mm or smaller are rarer because they are difficult to make of uniform dosage levels efficiently and cost effectively. This is due to the size and shape of the particles of the corresponding pharmaceutical powder blend as compared to the size of the die cavities in a punch-die configuration for making tablets. Regardless the disclosures of the above noted patents, typically, the prior art tablet tooling presents die cavity openings of constant surface area (i.e. a circular opening) so that orientation of irregular shaped particles is critical, and the Arching Index of the powder is critical. Due to such irregular shapes, many of the pharmaceutical or related material particles as prepared in practice have at least one dimension that is greater than that of the Arching Index dimension. In this case, the particles have a high tendency to bridge or arch over a die cavity opening and thus not enter the die cavity. The particles may in fact jam the die cavity opening, resulting in non-uniform tablets, which is not acceptable. The dosage will vary accordingly among the different capsules. The properties of such non-uniform tablets are not acceptable for further downstream process such as coating and filling into capsules or pouches. Also the irregular shape does not fill the cavity uniformly resulting in undesirable variation in medicament content from tablet to tablet.

A wurster column is a term of art for pharmaceutical formulation scientists. It is a special apparatus that allows for very efficient coating of small particles in a fluid bed dryer. It is commonly available on the market and known to those skilled in the pharmaceutical formulation arts. It uses a wet process to produce pellets by what is known as a drug layering technique Complex Perfect Sphere, CPS, is a technology offered by a company called Glatt that is a wet direct pelletization process, and is different from extrusion, but similar to drug layering that allows for production of pellets. It is known to one skilled in the art of pharmaceutical formulation as similar wet techniques such as rotor pelletization.

Roller compaction is used to form dry granules that can be compressed into tablets and comprises forming a compressed sheet from powders. The sheet is then later milled to produce free flowing granules. See the above noted '843 patent, which refers to pellets made with a moist process, but refers to the product as tablets. The term tablets as used herein refers only to dry powder and not to products made with solutions or wet processes, which is referred to herein as a pellet making processes. Milling is used to make the powder as noted in the '843 patent. The '428 patent also refers to a wet process using water that does not make tablets as defined herein, i.e., products made with a dry powder process.

It is rare that pellets (small diameter objects of about 1-2 mm diameter or smaller) can be made from the roller compaction and milling process, the properties of the compressed sheet and the subsequent milling of the sheet are not sufficiently reliable for this purpose. The particles produced by milling of the sheets are too angular and non-spherical to accept coatings readily.

Slugging is a process where very large tablets are made from a powder blend and then subsequently milled to produce smaller particles. It is unlikely pellets that are suitable substrates for coating could be produced by this process. Processes involving milling typically produces particles with undesirable angles, i.e., particles with sharp edges that do not resemble spheres and do not form acceptable substrates for subsequent coating.

Also, flat sides produced by the milling process necessary in roller compaction and slugging are subject to a phenomenon in the coating arts known as twinning, where the flat sides of two particles come into contact in the coating process and wind up sticking together forming a larger particle with a weak point where the two particles are held together by the coating. If the weak point breaks in later handling, the interior contents of the core becomes exposed, the coating will not be uniform and will not function as intended.

The sharp angles of irregular shaped particles such as those from a milling process present challenges for coating. It is difficult to achieve a sufficient coating on the apex of a sharp angle. This creates a weak point in the film covering the particle. It may be a point where the film is especially thin, thus requiring additional coating and reducing efficiency. Furthermore, if the coating is brittle, it may break off of the apex after handling for normal downstream process like capsule filling and expose the core contents so the then the film coating cannot perform the intended function. Still further the apex may break off in handling prior to or during the coating operation thus exposing the core contents or severely weakening the film.

None of the prior art processes are believed to be able to produce tablets of this small a dimension commercially. Pellet processes produce granules or pellets directly, and undergo no dry compression step in their formation. They require water or some other solvent. The present inventor contemplates a need for small tablets, e.g., about 1 mm diameter, to be produced in commercial quantities and in which products moisture is detrimental and must be avoided. Commercial punch and die manufacturers that provide punches and dies for pharmaceutical tablet production of the sizes discussed have problems as discussed above or in the '521 patent wherein the powders do not flow into the mating dies consistently in a desirable manner.

For example, one well known supplier of tablet forming equipment is The Elizabeth Companies. It has an internet site at www.eliz.com/compression.php. This site illustrates numerous punch and die equipment for tablet manufacture. None are useful for the manufacture of small tablets as discussed. Also single punch and die sets for producing single tablets are not desirable as they are not as useful for producing large quantities of tablets. In this case, such punch and die sets may be typically used in a rotary system employing numerous such sets in an annular array. See the literature in the above noted web site for example. Punch and die sets also include punches which produce multiple tablets simultaneously with a single die. See the above noted web site However, these sets use discrete punch and die sets for single tablets even though a given punch might have multiple tips for simultaneously producing multiple tablets with mating punches and die. The tablets are produced by discrete separate punch tips mounted on a single punch shaft. The problem with discrete punch and die sets (each punch and die produces a single tablet in a given compression cycle) for 1 mm diameter tablets is that they are relatively fragile due to their small dimensions and tend to break up during use. As a result, the punch and dies for 1 mm tablets have a very short life that is not commercially viable.

FMC corporation has also produced a manual related to formation of tablets and the associated tooling. Described are the punch and dies used among other detailed descriptions including rotary presses and the like. One of such rotary presses is described herein in connection with FIGS. 30 and 31.

A further commercially available rotary tablet filling machine is known as Comprima high speed tableting machine available from Industria Macchine Automatiche (IMA), Ozzano Emilia, BO, Italy, and as described in an article Centrifugal Die Filling System in a New Rotary Table Machine, P. L. Catellani et al. International Journal of Pharmaceutics, 83 (1992) 285-291. This machine is described in connection with FIGS. 32 and 33 herein. This machine has a rotary turret in which a hopper is filled with medicament powder. The powder is fed laterally to a plurality of stations comprising sets of an upper punch and lower die surrounding the hopper. The hopper is connected to the upper punches and lower punches by conduits each arranged horizontally about the hopper and extending radially laterally outwardly to the cavity formed by and between the upper and lower punches of each set. Centrifugal force flows the powder radially outwardly into the cavities. The upper punches are then lowered in a compression stroke to form the tablets which are then ejected after formation. However, the upper and lower punches of such a machine are also limited with respect to the size of tablets that can be formed and such upper and lower punches are not capable of forming tablets of about 1 mm in diameter in a commercially viable manner for same reasons discussed above.

The present inventor recognizes the above problems with the prior art punches in producing tablets of microminiature sizes in the range of up to about 1.5 mm in any transverse dimension of a tablet. The present inventor recognizes the source of the problem is that the prior art cavities and mating punches are each dimensioned to produce a discrete separate tablet. Such cavities of the dies for such discrete miniature tablets are too small to accept particle sizes of medicaments typically used in such tablets by commercially available practical processes without using additional costly particle processing steps.

The present inventor provides a solution to this problem by providing a tablet forming die cavity according to the present invention that is sufficiently large to accept particles of any random size that are commercially produced for such tablets regardless the Arching Index of the particles in cost effective powder producing processes without costly further particle processing steps.

The solution provides mating upper and lower punches that are configured to form the tablets into an interconnected tablet array in the particle filled cavity which is sufficiently large to accept all particles regardless their dimensions and Arching Index. It is the configuration of the punches that create the tablets such that when the punches enter the die cavity, they form an array of interconnected tablets with weakened interconnection regions between adjacent tablets.

The interconnections at the weakened regions then subsequently are easily readily severed to form the discrete tablets with applied force or forces such as by vibratory apparatus or the like.

A tablet configuration according to an embodiment of the present invention comprises an array of interconnected tablets and an array of links each forming an interconnection between each adjacent pairs of tablets, and which interconnections form weakened regions.

In one aspect, the plurality of tablets have a given composition, the links having the same composition as the tablets.

In a further aspect, the tablets generally may be any one of discs, spherical, generally in the shape of a pyramid, generally in the shape of mirror image pyramids, generally square in plan view, rectangular in side elevation view and wherein the tablets may have a first height h and the links have a thickness t, the height h being greater than the thickness t, or the tablets have a first height h and the links have a thickness t, the height h being the same as the thickness t.

In a further aspect, the links are dimensioned to form the weakened regions sufficiently weak so that the tablets separate from each other in the presence of an applied force or forces and preferably wherein the applied force or forces are induced by vibrating the tablet array.

Preferably, the tablets are circular and are interconnected to each other in tangential regions, the tangential regions forming the links.

In a further aspect, the links and tablets have a respective width dimension w_{L} and w_{T} the width dimension w_{L} of the link being less than that of the tablet w_{T}.

In a still further aspect, the links have a length dimension defined by the spacing between the tablets of the array of 0 mm to a value greater than 0 mm.

Preferably the links each have a transverse cross sectional area less than any transverse cross sectional area of any of the tablets.

In a further aspect, the tablets are identical.

A punch for producing tablets according to an aspect of the present invention comprises a shank and a punch tip attached to the shank. The punch tip has a base and at least one stanchion extending from the base. The at least one stanchion defines an array of cavities each corresponding to a tablet having a body formed by the cavities and an array of passages interconnecting the cavities for forming links interconnecting the array of bodies.

In a further aspect, a die is provided for use with a pair of punches as defined above and for further defining the array of cavities with the at least one stanchion.

In a further aspect, the die has a single cavity for producing an array of interconnected tablets in cooperation with the at least one stanchion of the pair of punches.

In a further aspect, a tablet body is defined between the stanchions having a transverse width w of no more than about 1.5 mm.

Preferably, the stanchions and base cooperate to define a generally circular cylindrical tablet body, or a portion of a generally square tablet body, or a generally pyramidal tablet body, or a portion of a generally spherical tablet body, or a generally rectangular tablet body.

A tablet forming punch and die set for forming an array of tablets according to a further aspect comprises an upper tablet punch, a lower tablet punch, and a die having a cavity cooperatively receiving the upper and lower punches. The die and punches are arranged to form a tablet of given transverse and height dimensions. dimensions. The punch and die are further arranged to configure the cavity to receive pharmaceutical powder particles that form the tablet. The arching index of the particles is greater in at least one dimension than the given transverse and height dimensions. Preferably, the die cavity is arranged to form a plurality of tablets.

In a further aspect, the punch and die are further arranged to configure the cavity to receive pharmaceutical powder particles that form the tablet. The arching index of the particles is greater in at least one dimension than the tablet given transverse and height dimensions. Preferably, the die cavity is arranged to form a plurality of tablets.

In a still further aspect, the invention includes a tablet made with the punch and die set described above.

In a further aspect, a method of forming tablets comprising forming an array of interconnected tablets having weakened regions between adjacent tablets.

In a further aspect, the method includes forming the array into a plurality of separate discrete tablets by breaking the array apart at each of the weakened regions.

In a further aspect, a force is applied to the weakened regions of the array to separate the tablets into discrete individual tablets.

In a further aspect, the step of forming the tablet comprises forming a cavity sufficiently large to form the array, filling the cavity with particles forming the array, and then compressing the particles to form the array.

In a still further aspect, the method includes forming a cavity larger than any dimension of any tablet of the array, filling the cavity with particles forming the tablets, and then compressing the filled cavity to form the array.

In a further aspect, the array is broken at each interconnection of the tablets to form the array into separate discrete tablets.

In a further aspect, the interconnections are removed from each tablet to form the interconnected array into a plurality of discrete separate tablets.

Each of the tablets is preferably in the range of up to about 1.5 mm in any dimension across the tablet.

In a further aspect, the invention includes a tablet made by any of the methods described above.

### IN THE DRAWING

FIGS. 1-5 are isometric views of tablets produced by apparatus according to various embodiments of the present invention;

FIGS. 6 and 7 are respective plan and side elevation views of a tablet array produced by apparatus according to an embodiment of the present invention in an intermediate stage of formation of the tablet of FIG. 3;

FIG. 6a is a fragmented side elevation view of the tablet array of FIG. 6 taken at lines 6-6;

FIGS. 8, 10 and 12 are respective isometric views of tablet arrays of intermediate stages of tablet formation corresponding to the tablet of FIG. 4;

FIG. 8a is a fragmented side elevation view of the tablet array of FIG. 8;

FIG. 9 is an isometric view of a tablet array of an intermediate stage of tablet formation corresponding to the tablet of FIG. 5;

FIGS. 10a, 10b and 10c are fragmented plan view diagrams of portions of a tablet array corresponding to the array of FIG. 10 showing different embodiments;

FIG. 11 is an isometric view of a tablet array of an intermediate stage of tablet formation corresponding to the tablet of FIG. 1;

FIG. 11a is a fragmented side elevation view of a portion of the array of FIG. 11 showing the link between adjacent tablets;

FIG. 12a is side elevation view of the array of tablets of FIG. 12 taken at lines 12-12;

FIGS. 13 and 14 are respective isometric and side elevation views of an intermediate stage of tablet formation corresponding to the tablet of FIG. 2:

FIG. 15 is a side elevation view partially in section of a punch and die set used to make the tablet array of FIGS. 6 and 7;

FIG. 16 are different isometric views of associated respective upper and lower punches and partially in section view of a die used to make the tablet array of FIG. 8 forming disc shaped tablets;

FIG. 17 is a plan view of the die of FIG. 16;

FIG. 18 are different isometric views of associated respective upper and lower punches and sectional view of a die used to make the tablet array of FIG. 8

FIG. 19 is an isometric sectional view of the die of FIG. 18;

FIG. 20 is an isometric view of the die of FIG. 18;

FIG. 21 is an isometric view similar to that of FIG. 16 except the punches are for creating spherical tablets as shown in FIG. 9 instead of the disc shaped tablets of FIG. 8;

FIG. 22 is a fragmented sectional elevation view of the punch surface portion of a punch of the punch array of FIG. 21 taken at linens 22-22 and used to make spherical tablets;

FIG. 23 is a plan view of the die of FIG. 21;

FIG. 24 is an isometric view of a die used to make the tablet array of FIG. 10;

FIG. 25 is a side elevation sectional view of the die of FIG. 24 taken at lines 25-25;

FIG. 26 is a top plan view of a lower punch used with the die of FIG. 24, there being an upper punch of the same configuration;

FIG. 26a is a fragmented isometric view of a divider section of an array of such sections of the punch of FIG. 26 forming the cylindrical shape of the individual tablets of the tablet array of FIG. 10;

FIG. 27 is a fragmented side elevation sectional view of the punch of FIG. 26 taken at lines 27-27;

FIG. 28 is a side elevation sectional view of upper and lower punches and die set corresponding to the die of FIG. 24 and punch of FIGS. 26 and 27:

FIG. 29 is a side elevation sectional view of a punch and die set used to make the tablet array of FIGS. 13 and 14;

FIG. 30 is a side elevation diagrammatic representation of a commercially available rotary tablet press apparatus useful for making tablets with the punch and die sets of the present invention;

FIG. 31 is a top plan view of the apparatus of FIG. 30;

FIG. 32 is a sectional elevation diagrammatic representation of a commercially available centrifugal rotary tablet press apparatus useful for making tablets with the punch and die sets of the present invention; and

FIG. 33 is a sequence of fragmented side elevation sectional views of a representative punch and die set of FIG. 32 showing steps a-f for forming a tablet.

### DEFINITIONS OF TERMS USED HEREIN

Arching Index—A particle dimension or a dimension of the cohesion of multiple pharmaceutical particles to each other to form a larger unitary cohesive particle during the die filling process of a tablet forming process, the dimension being relative to the diametrical or transverse extent across a tablet forming die cavity opening or the depth or any other dimension of the cavity receiving the particles. The Arching Index dimension may be greater than any dimension of a tablet.

Tablet—A solid dosage form or partial dosage form, with or without a coating, of dry compressed pharmaceutical particles formed with no moisture present.

Pellet—A dosage of pharmaceutical particles formed with a wet process.

Die—A device having a cavity for simultaneously forming one or more tablets.

Punch—A device that cooperates with a die and a second punch for simultaneously compressing pharmaceutical particles into one or more tablets.

Punch and Die Set—A pair of punches which mate with a die for forming one or more tablets.

Link—A structure for interconnecting a pair of adjacent tablets having a length dimension extending to the adjacent tablets ranging from at least 0 mm to some finite dimension.

Pharmaceutical Particle—A particle used to form a pharmaceutical dosage unit.

Pharmaceutical Dosage Unit—The amount of an active pharmaceutical substance (with or without inactive ingredient(s)) administered to a person or animal.

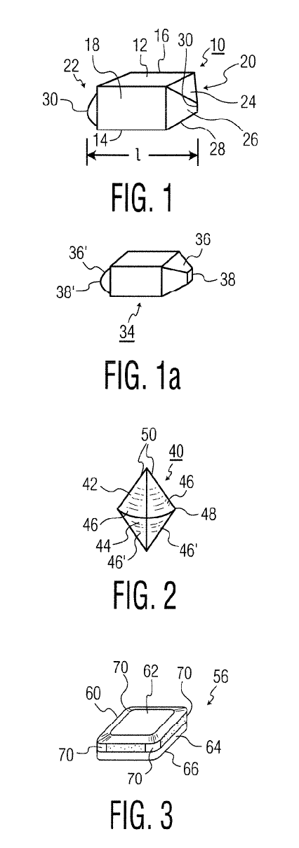

FIGS. 1-5 illustrate examples of tablets that are made by the apparatus and processes according to various embodiments of the present invention. After the tablets are described, the specification below will discuss the various tools to fabricate these tablets.

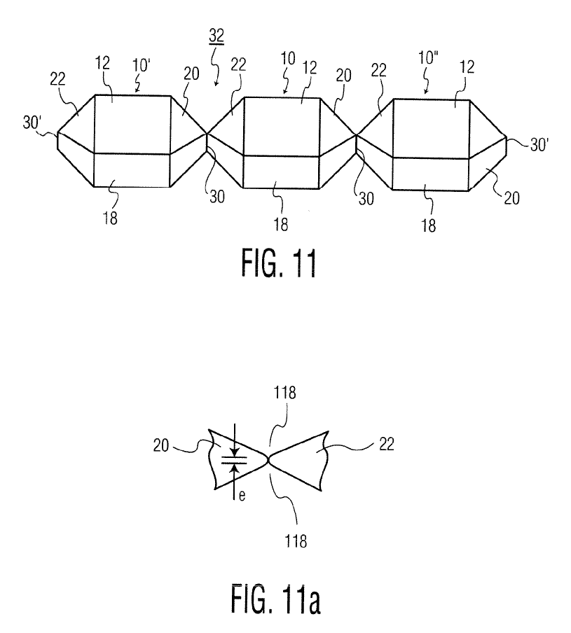

In FIG. 1, tablet 10 has rectangular top and bottom surfaces 12, 14 (which also may be square) and rectangular opposite side surfaces 16, 18 (which also may be square). Each end of the tablet 10 has preferably identical frusto-pyramid segments 20, 22. Representative segment 20, in the case where surfaces 12-18 are identical rectangles or identical squares, has identical inclined sides 24, 26, 28 and so on. Each side terminates at end surface 30 which is an elongated edge. The tablet opposite end surfaces 30 are created by fracturing a weakened web region e, FIG. 11a, that was interconnected to an adjacent segment 22 of an adjacent tablet 10 as best seen in FIGS. 11 and 11a.

In FIG. 11, the tablets 10, 10′ and 10″ are formed as a monolithic interconnected linear array 32. The tablets 10, 10′ and 10″ are interconnected by a link of the same composition as the tablets at severed surface 30 formed by a web at region e, FIG. 11a, between adjacent segments 20, 22. There may be three or more such interconnected tablets in the array, the three tablet array of FIG. 11 being exemplary. The end tablets 10′ and 10″ have segments 20 and 22 respectively, but these were never interconnected and their respective edges 30′ and 30″ are formed by pressure punching in a mating die and not by severing. The intermediate edge surfaces 30 between adjacent tablets 10′ and 10 or 10 and 10″ are formed by severing the web interconnection between the segments 20 and 22. This web interconnection is a linear edge.

In FIG. 1a, the tablet 34 has opposite end segments 36, 36′. These end segments terminate in severed end surfaces 38, 38′, respectively, except for the end tablets of the linear array (not shown). These end surfaces are molded by the pressure in the die and not formed by fracturing. The end surfaces 38, 38′ were originally fractured weakened webs that interconnected a plurality of adjacent tablets 34 in the linear array (not shown). This array is similar to the array 32, FIG. 11a.

The tablet 40, FIGS. 2, 13 and 14, is bi-pyramidal, i.e., it has two mirror image pyramid like portions 42 and 44 extending in opposing directions. The upper portion 42 has four like sections 46. The lower portion 44 similarly has four like sections 46′ which are the same as sections 46. The sections 46 and 46′ have a circular junction 48 which is an edge. Adjacent sections 46 terminate at a junction forming a linear inclined edge 50 to form a pyramid-like shape. In the alternative, the sections may not terminate at an edge 50 and instead may form a cone.

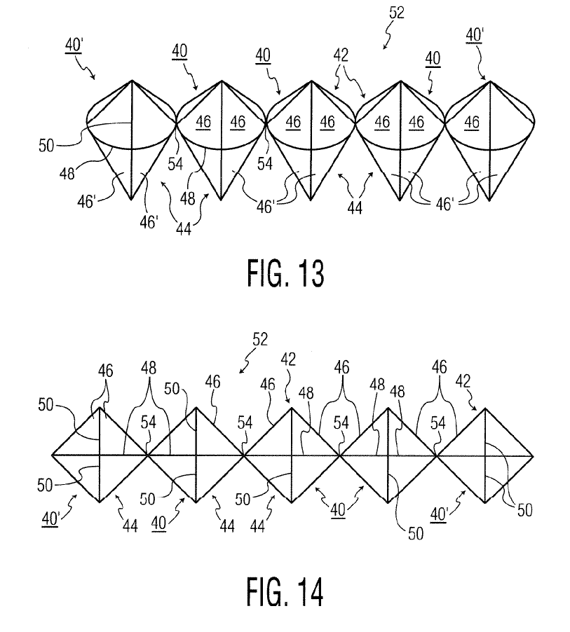

The tablets 40 are formed in a punch and die set (not shown), but in a manner to be explained with respect to others of the tablets described herein, to form a linear array 52 of tablets, FIGS. 13 and 14. The next adjacent tablets are joined monolithically by interconnection web-like links 54 at the tangential regions of junctions 48 to form a unified structure of a given composition. The web-like links 54 form weakened regions that readily fracture when stressed such as may be induced by vibrations or the like. This fracturing separates the tablets into separate discreet tablets at fracture locations at the weakened regions forming tablets such as tablet 40, FIG. 2. The interconnection links 54 represent small areas that are relatively weak, so that the tablets readily separate when subject to stresses induced for example by a vibratory apparatus of the type that is commercially known. As discussed in the introductory portion, edges and points are not desirable for coated tablets, so the tablet 40 is not as desirable as the other tablets described herein.

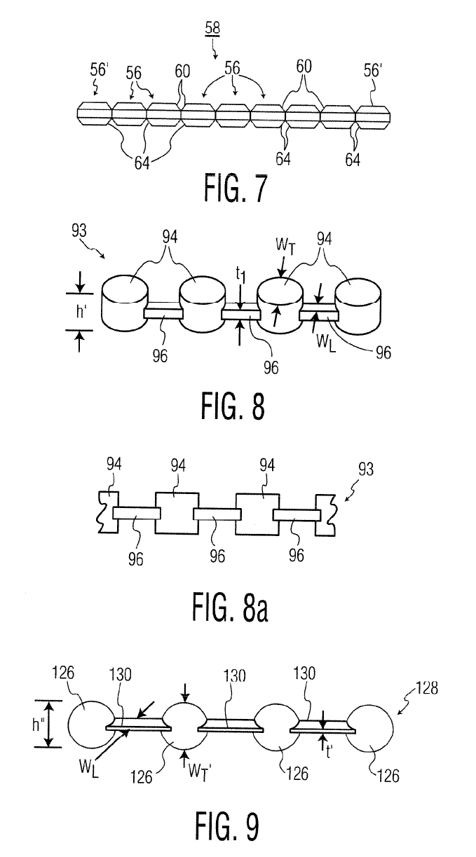

In another embodiment of the tablets, tablet 56, FIGS. 3, 6 and 7, is generally square in plan view as seen in the array 58 of tablets 56, FIG. 6. Representative tablet 56, FIG. 3, has a first chamfer 60 surrounding a flat plateau 62 on the tablet top and a second chamfer 64 surrounding the flat plateau on the tablet bottom. An outer peripheral surface 68 surrounds the tablet between the upper and lower chamfers and forms a side wall normal to the plateaus, FIG. 7. The corners 70 of the peripheral surface 68 are rounded, e.g., by radii

In FIG. 6, the tablets 56 are formed of a common composition in a monolithic array structure including array edge tablets 56′. These tablets are formed in a generally square array 58. The surfaces between the corners 70 are planar and are joined in monolithic fashion to adjacent ones of the tablets to form a weakened joint or interconnecting link web 76 (FIG. 6a). The web 76, FIG. 6a , has a thickness d preferably of such a dimension that the web will readily fracture in the presence of vibratory induced forces. The web 76 is relatively thin in a direction normal to the thickness d dimension.

In this embodiment as in all of the embodiments, the tablet monolithic composition is such that the compressed material is relatively brittle. This material fractures readily in the presence of vibratory induced forces. The composition in some embodiments is one that is moisture sensitive and the particles must be dry at all times. Such dry particles have the undesirable variation in outer dimensions which makes it difficult for them to fill conventional dies of about 1.5 mm diameters. This composition can not be produced as small pellets as produced by a wet process of the type discussed in the introductory portion because of its moisture sensitivity.

When the web 76 is fractured, the tablet is left with a resulting surface 72 at the fracture, FIG. 3, of each adjoining tablet 56, 56′. In FIG. 3, the surfaces 72 are stippled and represent these fractured surfaces. Surfaces 72 are created by fracturing the web 76 joining the adjacent tablets of the array 58, FIGS. 6 and 6a.

In FIG. 6, the arcuate corners of the tablets result in open somewhat diamond shape spaces 74, separating the weak interconnecting webs 76 at the junction of the adjacent tablets 56. 56′. The webs are fractured by subjecting the array to forces such as induced by vibrations and the like. This forms the array into a plurality of separate discrete tablets 56, FIG. 3. Tablets 56, 56 ′ are preferred.

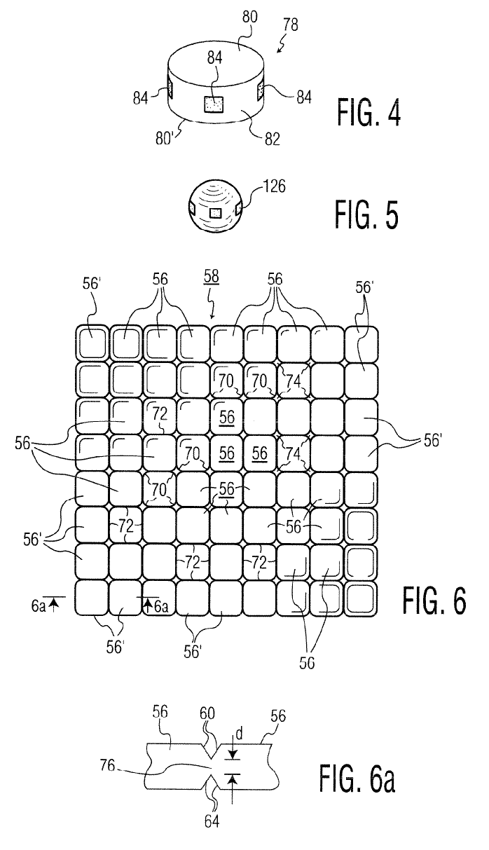

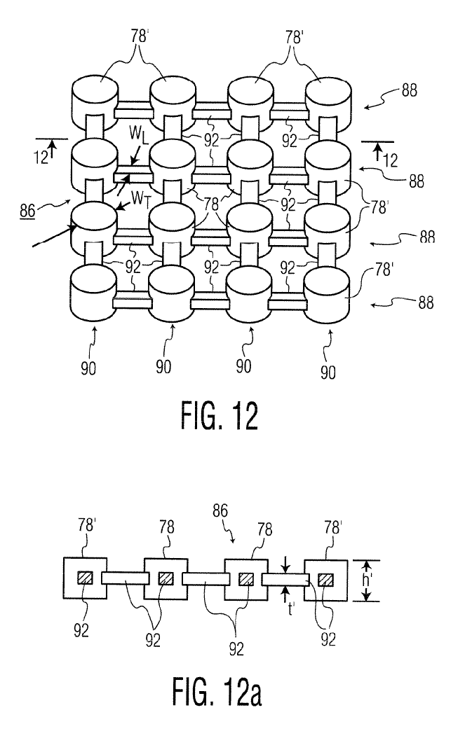

Another preferred tablet is tablet 78, FIG. 4. This is disc shaped and is circular cylindrical. This tablet has flat circular top and bottom surfaces 80, 80′, ′ respectively, a circular cylindrical side wall 82. In the alternative, the top and bottom surfaces may be somewhat convex (not shown). Portions 84 of the side wall 82 are square (or rectangular in the alternative) fracture surfaces as represented by the stippling. The tablets 78 are initially formed in a matrix array 86, FIGS. 12 and 12a, comprising rows 88 and columns 90 of linear arrays of four tablets each. Tablets 78′ form the peripheral outer arrays of the matrix array 86 and tablets 78 are interior the array 86. The array 86 is formed by a corresponding punch and die set as will be explained.

The tablets 78, 78′ are interconnected by links 92, which preferably are identical. The links 92 and tablets are monolithically formed of the same composition. The links 92 may be rectangular, square or round in transverse cross section. In this embodiment the links 92 are square in cross section as shown in FIG. 12a. The links 92 have a thickness t′, FIG. 12a, and a width w_{L}, FIG. 12. The tablets have a width (the diameter) w_{T}. The link widths are less than the tablet widths. The link thicknesses are less than the height h′ of the tablets, FIG. 12a. The tablets preferably have a diameter of about 2 mm or less and a height of about 1.5 mm or less. The links 92 thus are a fraction of a mm in transverse width and thickness. Because the composition of the links is brittle after compression, they readily fracture in the presence of forces such as induced by vibrations on a vibratory apparatus. Preferably, the links fracture at their junctions with the associated tablets to form the fracture surfaces 84, FIG. 4. If the links break intermediate the tablets, the vibratory forces are such as to disintegrate the protruding link portions due to their brittleness. In this case, the tablets may be subject to tumbling forces in a rotating drum to fracture and disintegrate the links 92. The relative robust strength of a cylindrical tablet as compared to the protruding brittle links precludes the tablets from fracturing when tumbled or otherwise subject to vibratory or other forces. Such removal of the links and separation of the tablets without damage thereto can be determined empirically for a given set of tablet and link parameters by one of ordinary skill. The disintegration of the links produces a powder which can be removed by a deduster as known in this art. Preferably the cross section and length dimensions of the links 92 are set to those values so that the tablets are readily separated and the links reduced to powder without damage to the tablets. This is true for all of the tablet embodiments discussed above and their associated arrays and links or interconnecting webs.

In FIG. 8, in an alternative embodiment, a linear array 93 of tablets 94 is shown. The tablets 94 are circular cylindrical discs or cylinders as discussed above in connection with the embodiment of tablets 78 of FIG. 4. The difference is that the tablets 94 are formed in a linear array. FIGS. 8 and 8a, rather than in a matrix array as in the embodiment of FIG. 12 of tablets 78. The tablets 78 and 94 are generally the same as described above.

The tablets 94 are interconnected by links 96 in the linear array, which links 96 may be identical to the links 92, FIGS. 12 and 12a. The tablets 94 have a diameter w_{T} and the links 96 have a width w_{L} and a thickness t₁ These dimensions are related similarly as the corresponding dimensions discussed above in connection with FIGS. 12 and 12a.

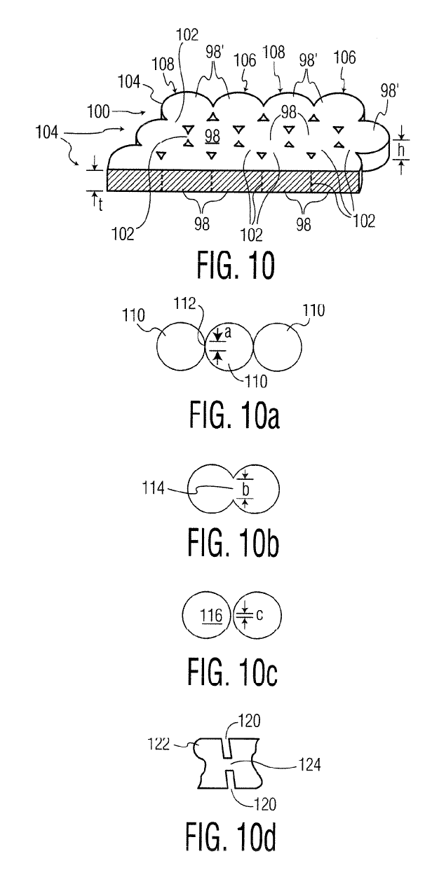

In FIG. 10, an embodiment of a disc tablet, tablet 98, similar to tablet 78 of FIG. 4, is shown. The difference is that tablet 98 is formed in an array 100, FIG. 10, that is different than the prior arrays of tablets corresponding to tablet 78 discussed above. The tablets 98 are interior the array 100 whereas the tablets 98′ are at the outer edges of the array 100. The tablets 98, 98′ are circular cylinders that are joined tangentially to each other at an annular array of locations forming interconnection links 102 between the adjacent tablets. The interior tablets are tangential with six adjacent tablets at corresponding six tangential interconnecting links 102, shown as dashed lines in the sectional portion of FIG. 10, which interconnections form interfaces or webs between the adjacent tablets. Only a portion of the array 100 is shown. The array is formed as a matrix of tablets wherein the tablets in adjacent rows 104 form staggered columns 106 and 108 in alternating fashion. An array of tablets such as tablets 98, 98′, according to another embodiment, may be formed into the shape as shown by way of example by the punch tool of FIG. 26.

In FIGS. 10a, 10b and 10c, various embodiments of the tablets of FIG. 10 are shown. In FIG. 10a, tablets 110 of one array of tablets is illustrated. This array is only partially shown as three tablets These tablets represent a much larger array such as the array of FIG. 10 or the array as produced by the tool of FIG. 26. FIG. 10a is presented to illustrate certain principles of the interconnection links, such as links 102 of FIG. 10. In FIG. 10a, a link 112 has an interface with the adjoining tablets of dimension a. In FIG. 10b, the link 114 has a wider dimension b. In FIG. 10c the link has an interface of dimension c which is smaller than dimension a of FIG. 10a. The dimensions a, b and c are determined empirically to ascertain that value which maximizes the separation of the tablets when exposed either to tumbling or vibratory forces and the like.

In addition. the links 112, 114 and 116 of the respective FIGS. 10a, 10b and 10c have a thickness dimension corresponding to dimension t′ of FIG. 12a, which may be smaller than the tablet height dimension h′. In this case, the smaller dimension of the interface than the height dimension h′ produces an interface between adjacent tablets somewhat similar to that shown by dimension e in FIG. 11a. However, the interface may be formed by opposing rectangular channels in the interface between adjacent tablets, FIG. 10d, rather than the opposing V-shaped channels 118 as shown in FIG. 11a (and also in the embodiment of FIG. 6a). The rectangular channels then produce interconnecting links corresponding to links 102 of FIG. 10. These are of reduced dimension as compared to the tablet 98, 98′ height dimension h.

FIG. 10d illustrates rectangular channels 120 between tablets 122 forming an array corresponding to the array 100, FIG. 10. The rectangular channels 120 form weakened regions at interconnecting links 124, which facilitate separation of the tablets by vibratory or tumbling forces and the like with a minimum of residual material left in the links 124 interconnecting the tablets. The channels 120 in this case have a transverse width (left to right in the figure) significantly smaller than 1 mm, e.g., 0.01 mm or smaller.

The depth of the channels (from top to bottom of the drawing) is also determined empirically. The depth is such as to permit the mating rib on the punch, to be described, forming the channel, to be reliable. This rib is required to survive repetitive punch operations. The rib is of relatively small dimensions and may be required to withstand high pressing forces. The compromise rib dimensions forming the channels 120 to maximize weakness of the link 124 and also that of the reliability of the mating punch for high numbers of repetitive punch operations can be readily determined by one of ordinary skill.

In FIG. 5, an alternative spherical tablet 126 is illustrated. This tablet is formed by a linear array 128, FIG. 9. The tablets 126 are interconnected by links 130. The links 130 have a width dimension w_{L} and a thickness t′ which dimensions are the same as discussed above in connection with the tablets of the embodiment of FIG. 8. The spherical tablets have a diameter h′ and is related to the link dimensions similarly as the height h of the other embodiments discussed above. Also the various linear arrays may be arranged as matrix arrays such as shown in FIG. 12 for all tablet types described herein.

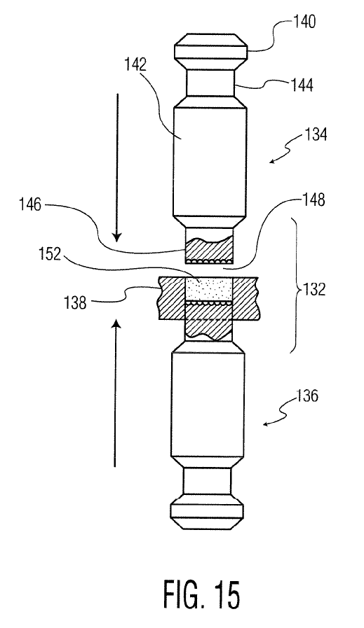

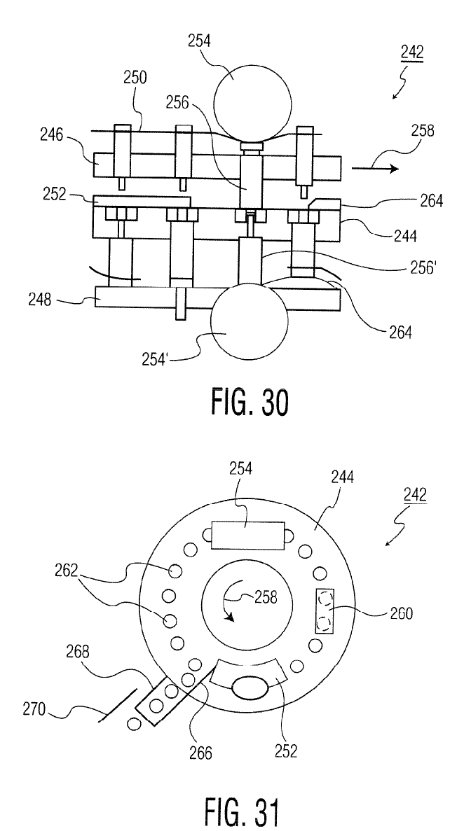

The tooling for producing the various tablets will now be described. In FIG. 15, punch and die set 132 comprises an upper punch 134 and a lower punch 136, which punches are identical. A die 138 is between the two punches. The die 138 is preferably mounted on a rotary table forming apparatus as will be described, for example, in connection with FIGS. 30 and 31 below.

Upper punch 134 is representative and will be described, it being understood that lower punch 136 is the same in construction. Upper punch 134 is made of tool steel as is conventional in this art. The punch 134 has a head 140 and shank 142 of conventional design. The head 140 is connected to cylindrical shank 142 that has a reduced diameter annular channel 144 for attachment to the mating apparatus operating the punch 134. These structures being conventional need not be described further.

The shank 142 terminates at its lowermost end opposite the head 140 in a reduced transverse dimension operating punch tip 146. The tip 146 terminates at its lowermost end region 148 in a three dimensional configuration shaped to form the tablet array 58, FIGS. 6 and 7. This end region 148 mates with an identical working tablet forming three dimensional end region of lower punch 136 to form the array 58 of tablets. The end regions 148 of the two punches are aligned with and face one another. These end regions have an array of complementary recesses which form the tablets 56, FIGS. 6 and 7.

The die 138 has a cavity 150. The cavity 50 is fed particles 152 forming the tablets 56 by apparatus not shown, but well known in this art. The cavity 150 has a cross sectional area sufficient to closely receive the tips of the two punches 134 and 136. The lowermost punch 136 is first inserted into the cavity 152 to its operating position as shown in FIG. 15 prior to filling the cavity with the particles. The particles are then dispensed into the cavity 152 with the lower punch in place.

As discussed in the introductory portion, the particles may have dimensions or Arching Index larger than any dimension of the tablets 56, FIGS. 6 and 7. As will be appreciated, the working end regions of the punches have recesses or cavities complementary to the shape of the tablets being formed. Since the particles or Arching Index may have a dimension larger than any dimension of the tablets, it is important that the recesses or cavities of the lower punch be filled to ensure that uniform density tablets are formed. If the particles fill the various tablet forming recesses or cavities to different volumes, then the resulting tablets will have different amounts of particles and the dosage unit among the different tablets will thus differ, which is undesirable.

This problem of filling the recesses or cavities uniformly is substantially resolved by the configuration of the array of tablets which are interconnected as described above. These interconnection links have mating recesses or passageways in the punch working surface region 148, FIG. 15. These cavity or recess interconnecting passageways effectively enlarge the recesses or cavities corresponding to and defining each tablet body. This cavity enlargement creates a cavity space for particles regardless of their arching indexes that are greater in dimension than the tablets. That is, for those particles or arching indexes longer than a transverse dimension of the tablet, a portion of that particle or arching portion of several particles based on their arching index will extend into the adjoining link forming recess which joins the adjacent tablets. Such links accommodate particle or arching portions that may extend beyond the tablet forming recesses of the punch Thus the tablet forming cavities or recesses in the punch are considerably enlarged. As such, the enlarged cavities or recesses tend to fill up more uniformly than would otherwise occur, e.g., if discrete recesses or cavities without the interconnecting link passageways were to be provided as in the prior art. This will be explained further in connection with the other embodiments of the tooling for the other tablets shown in more detail in several of the drawings.

Once the cavity 152 with the lower punch in place is filled, keeping in mind that the cavity is sufficiently large to accommodate all of the tablets produced by the punches, the upper punch is lowered and the filled particles are compressed in the die cavity to form the array 58 of tablets 56. The array is then processed in a vibratory or tumbling apparatus (not shown) to sever the web interconnections, i.e., the links, between the adjacent tablets formed by the connecting passageways. This creates separate discrete tablets 56 of the desired miniature dimensions of about 2 mm or less and preferably about 1.5 mm with a tablet height of preferably about 1 mm. Any protrusions caused by breaking the tablets apart are disintegrated by the vibratory or tumbling action. The resulting powder is then removed by conventional dedusting apparatus, which removes powder that resembles dust grains.

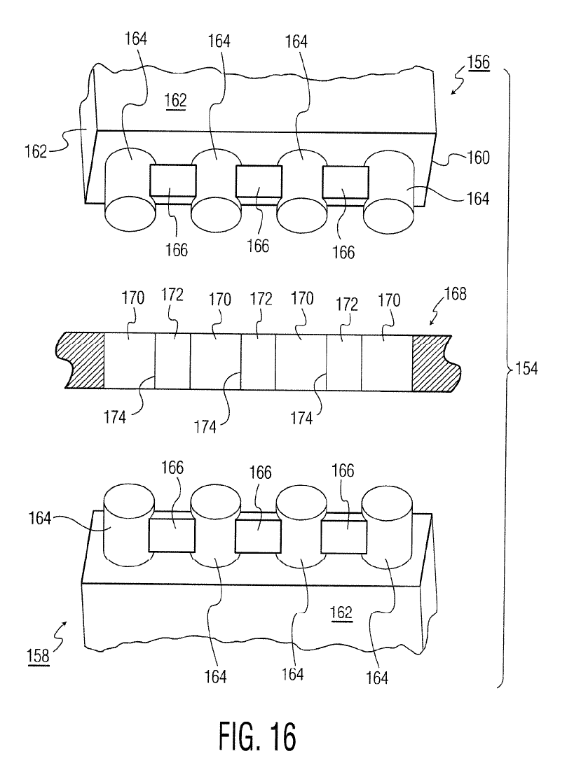

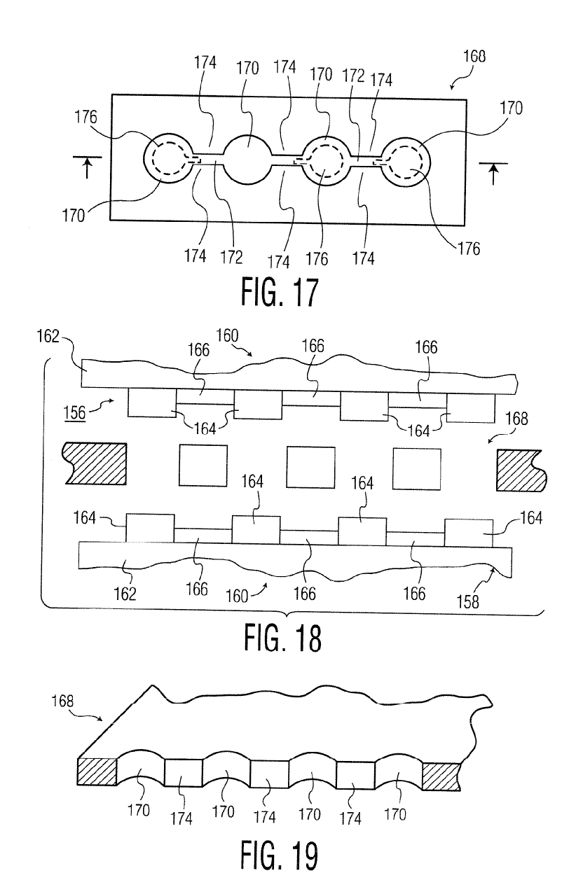

In FIGS. 16-17, punch and die set 154 fabricates tablet array 93, FIGS. 8 and 8a, which produces disc tablets 94, which are somewhat similar to the tablets 78, FIG. 4. The set 154 includes an upper punch 156 and a lower punch 158. The upper and lower punches are identical so a description of punch 156 is representative. The working tip portions of these punches as well in the subsequently described figures are illustrated in the various figures, the remaining punch portions being conventional as described in connection with the punches of FIG. 15.

In FIGS. 16 and 18, upper punch 156 has a lower tip working portion 160. The tip portion 160 comprises a shank portion 162 from which depends a linear array of spaced like male circular cylindrical punch segments 164. The segments 164 are interconnected by like bridging male link passageway producing punch segments 166. The segments 164 punch the powder particles into the tablet discs such as in tablets 94, FIG. 8, and the link punch segments 166 form passageways between the tablet forming cavities, and which passageways form the weakening region links, the interconnecting links 96, FIG. 8. The lower punch 158 is identical to the upper punch, is positioned in mirror image relation to the upper punch, and its punch segments cooperate with the identical mating segments of the upper punch to compress the powder particles to form the array 93, FIG. 8.

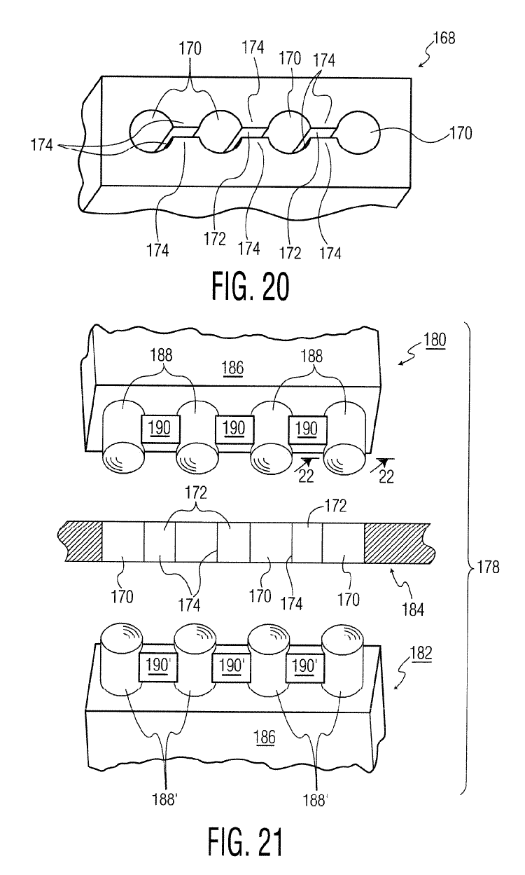

A die 168, FIGS. 16-20, is between the upper punch 156 and lower punch 158. The die 168 has an array of circular cylindrical cavities 170 interconnected by passageways or linear channels 172. The channels 172 are formed by a pair of like facing projections 174. The channels 172 form the mating weakening links 96 of the resulting tablet array 93 produced by this punch and die set, FIGS. 8 and 8a.

In operation, the lower die 158 male punch segments 164 and 166 are first inserted partially, e.g., about 50%, into the corresponding cavities 170 similar to the lower punch of FIG. 15. The segments 164 enter and engage the corresponding interconnecting passageway channels 172, all of which segments closely fit in the corresponding cavities and channels. The dry particles then are filled into the now enlarged cavities 170 via the passageway channels 172 with the lower punch in place.

Conventional particle filling devices are employed and need not be described herein. As shown in phantom in FIG. 17, particles 176, (or their arching index) which are larger than any transverse dimension of the identical cavities 170 (it being recalled the depth of the cavities with the lower punch in place is about 0.5 mm (reduced by about 50%) and the diameter of the cavity is in the range of about 1.5 to 2 mm), have portions thereof which fall into the passageway interconnecting channels 172. These passageway channels are formed by projections 174 and permit the larger particles to fill up the otherwise smaller cavities 170.

After the cavities and channels are filled, the upper punch is lowered, the particles are compressed and the array 93, FIG. 8, is formed. The tablet array 93 is then released from the die 168 in a known manner after the punches are removed from the die cavities. The tablets are then separated by applied forces as discussed above. When the tablets are separated, there may be negligible dimensioned links formed by the tangential regions as in FIGS. 10a, 10b and 10c when produced according to this embodiment In the alternative, there may be links of greater length, FIG. 8, if produced per this embodiment. When there are links of greater length, FIG. 8, the links are broken off the tablets and/or may be disintegrated into powder form (and if necessary, by apparatus not shown).

In FIG. 21, punch and die set 178 punches spherical tablets according to FIGS. 5 and 9. The set 178 includes respective upper and lower punches 180 and 182 and a die 184. The die 184 may be, and preferably is, identical to the die 168, FIGS. 16-20. The die 184 need not be described further for this reason.

The upper punch 180 has a lower tip working portion 186. The tip portion 186 comprises a shank portion from which depends a linear array of spaced like male circular cylindrical punch segments 188. The segments 188 are interconnected by like bridging male link producing punch segments 190 which form the interconnecting passageways between the adjacent tablets to be formed. The segments are not to scale, see FIG. 16, and in practice, may be smaller in relative depth than that shown (from the top of the drawing sheet to the bottom). The segments 164 punch the powder particles into the tablet discs such as in tablets 94, FIG. 8, and the link punch segments 166 form the weakening regions via the mating passageways in the die, interconnecting links 96, FIG. 8. The lower punch 158 is identical to the upper punch, is positioned in mirror image relation to the upper punch, and its punch segments cooperate with the identical mating segments of the upper punch to compress the powder particles to form the tablet array.

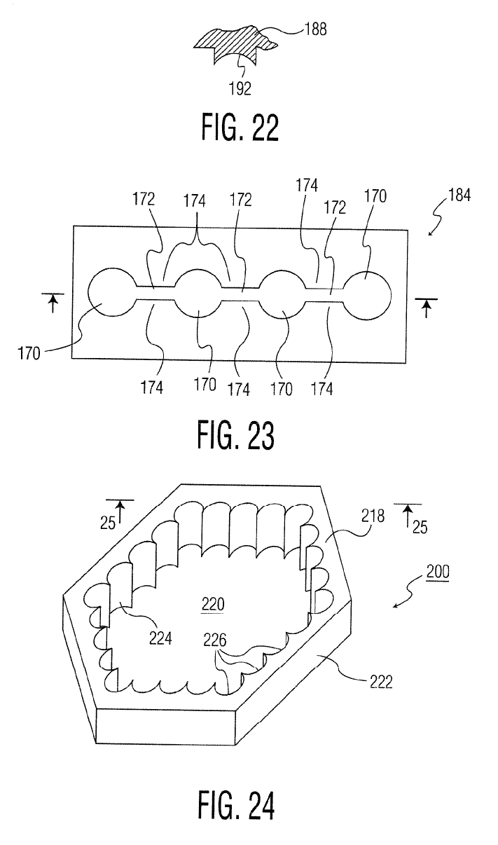

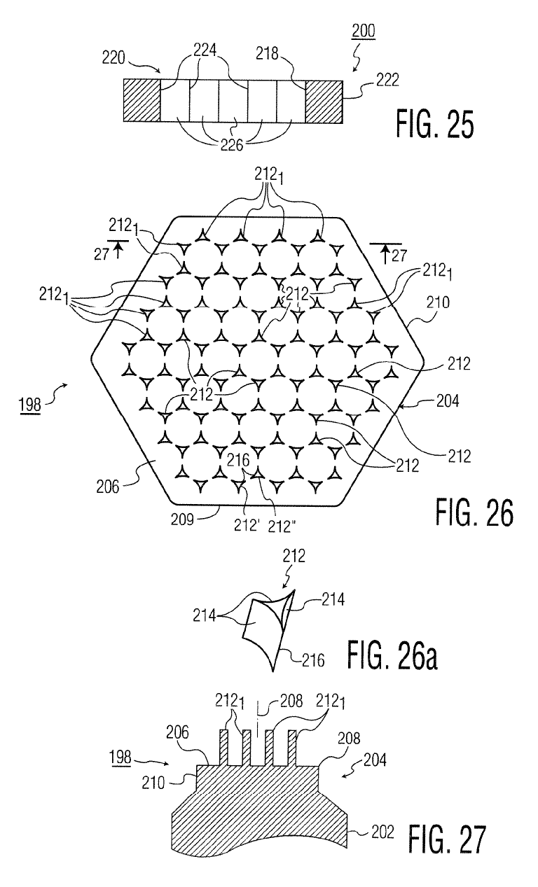

In FIG. 22, representative punch segment 188 has a semi-spherical concave cavity 192 for forming a spherical tablet in cooperation with the mating lower punch segment 188′, FIG. 21.

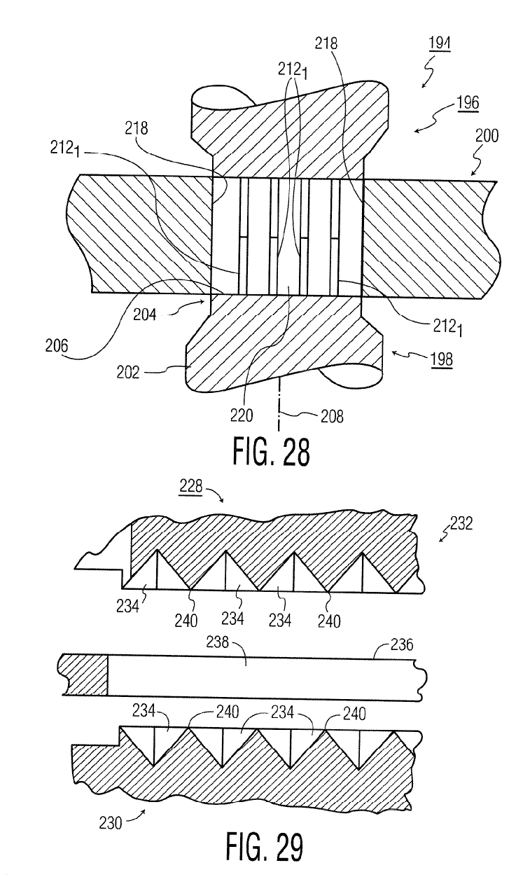

In FIG. 28, punch and die set 194 includes an upper punch 196, a lower punch 198 and a die 200. The set 194 produces the tablet array 100, FIG. 10. In this array the tablets 98 are interconnected by tangential regions as discussed above in connection with FIGS. 10a, 10b and 10c. Representative lower punch 198, FIG. 28, has a shank 202 and a tip portion 204. The punch tip portion 204 has a transverse planar surface 206 normal to the punch shank longitudinal axis 208. This surface 206 has a hexagonal peripheral edge 209 defined by hexagonal outer surface 210 of the tip portion 204.

A plurality of tablet array 100 forming stanchions 212 extend upwardly from the surface 206. The stanchions 212, FIG. 26a, are generally triangular in cross section, each side having an identical circular segment surface 214. The stanchions 212 are spaced in circular arrays of six stanchions along equally spaced radii emanating from a center point of each tablet being formed. A surface 214 of each of six stanchions 212 forms a circular portion of the outer periphery of each tablet.

The spacing of the stanchions is along the outer peripheral surface of the so defined cylindrical tablet. Six stanchions define each tablet and are arranged in equal hexagonal spacing about a center point. Adjacent stanchions such as stanchions 212′ and 212″, for example, FIGS. 26 and 26a, have an edge 216, normal to surface 206. Their respective edges 216 of all of the various pairs of stanchions are aligned on an imaginary outer circular periphery of a tablet being formed thereby The edges 216 define the ends of the tangential contiguous region between adjacent contiguous tablets forming an array 100 (FIG. 10). The array 100 of tablets, FIG. 10, is also hexagonal in shape.

The outer most stanchions 212₁ FIG. 26, cooperate with the mating die 200, FIG. 28, to form the outermost tablets. In particular, the outer most stanchions cooperate with the die cavity 220 formed by peripheral cavity wall 218. The upper punch 196, FIG. 28, being identical to the lower punch 198 together with the die 200 define and form the tablet array 100. The stanchions 212 of the upper and lower punches are axially aligned parallel to axis 208 as seen in FIG. 28. No powder is essentially between the upper punch and lower die stanchions so that the stanchions do not perform any significant compression step of the powder particles pressed by the two punches. The stanchions are sufficiently small in transverse dimension so as to not trap any significant portion of particles therebetween.

In FIG. 24, the die 200 has a cavity 220 as discussed above. The die 200 has a hexagonal outer wall 222, but this is arbitrary and could be any shape and preferably may be rectangular to simplify manufacturing cost and use in a mating rotary tablet forming apparatus to be described below. The wall 218 of the cavity 220 has a scalloped interior surface 224. The surface 224 is formed with an array of contiguous circular segment shapes comprising surface 224. Each concavity 226 of the surface 224 corresponds to a tablet of the array 100 Each such concavity 226 cooperates with the adjacent stanchions 212₁, FIG. 26, at the outermost regions of the array of stanchions 212 to form each tablet. There may be two or three such outer adjacent stanchions 212₁ for each outer tablet.

As seen in FIG. 24, the cavity 220 is considerably larger than any of the relatively smaller tablets of the array 100, FIG. 10, being formed thereby. This readily avoids the problem associated with filling the cavity with particles that may be larger than or have an arching index larger than any transverse dimension of the resulting tablets being formed.

Of course, the lower punch 198, FIG. 28, needs to be in place prior to filling the cavity 220 of the die 200. In FIG. 28, the lower punch only extends partially into the cavity at the cavity lower half portion. The particles fill the entire cavity prior to the upper punch being lowered into the cavity to compress the particles in the position of FIG. 28. The spaces between the stanchions is relatively open as seen in FIG. 26 and the upper half portion of the cavity 220 above the lower punch is clear. There are no transverse obstructions at all in the cavity between opposite sides of the cavity wall 218 above the lower punch 198. Thus, particles of any dimension larger than a transverse tablet dimension readily fill the cavity 220 prior to lowering the upper punch 196.

The punch and die set 194 is somewhat similar to the punch and die set 132, FIG. 15, forming the array of tablets of FIG. 6. The stanchions of the punches of FIG. 15 are shaped more as diamonds than triangles. The stanchions for the punch and die set 132 are arranged in a square array to form the generally square shaped tablets of the array of FIG. 6. The upper and lower punches appear somewhat similar to the punch and die set 194 of FIG. 28 in cross sectional view. The description of the set 194 applies generally to that of set 132.

FIG. 29 shows a punch and die set 228 for forming the tablet 40, FIG. 2, and the array of interconnected links and tablets 52, FIGS. 13 and 14. In FIG. 29, the set 228 comprises an upper punch 232 and an identical lower punch 230. The tip portion of the upper punch is representative. It has a linear array of like cavities 234 which are complementary to the cavities in the lower punch 230 and which cavities are negatives of the shape of the tablet 40, FIG. 2, being formed. The die 236 has a cavity 238 which receives the upper and lower punches.

As readily seen, the cavity 238 is relatively large compared to the transverse dimensions of the individual tablets being formed. The particles if larger than the cavities 234 of the lower punch 230 merely lie along the inclined sides of the cavities 234 and may extend into the upper portion of the die cavity 238. When the upper punch 232 is lowered it merely compresses the particles and pushes them into the punch cavities, if they are extending beyond and across the cavities. In the alternative, the punches having relatively mating sharp edges 240, may break up the particles if they happen to bridge adjacent cavities of the lower punch.

In FIGS. 30 and 31, rotary tablet press 242 comprises die table 244, an upper punch guide section 246 and a lower punch guide section 248. The die table 244 receives the associated dies of a punch and die set, e.g., die 138, FIG. 15, die 168, FIG. 16, die 184 FIG. 21 and die 200, FIG. 24 and so on. These dies fit in mating recesses in the table 244 The punch guide sections 246 and 248 receive, releasably secure and guide the punches as they traverse vertically from the quiescent positions to the tablet pressing positions.

The upper section 246 includes punch resilient sheet metal retainer device 250, which is a cam track that receives and returns the lowered punches to their upper position For example, the punch shank 142, FIG. 15, has an annular channel 144 which is received by device 250 for resiliently retaining the punch to the press 242. The guide section holds the punch assembly and guides the moving punch as it lowers and raises during operation. A particle filling station 252 is secured to the table 244. The operation of the filling station is conventional, well known in the industry and need not be described. For example, paddles (not shown) rotate within a feed frame to provide agitation. The agitation causes the powder particles to remain free flowing and thus readily flow into the respective die cavities.

An upper compression roller 254 causes the associated punch 256 to be lowered to a tablet compression position as the punches rotate beneath the roller 254. The upper punch follows the cam track to a position under the roller 254 where the cam track is discontinued and, after compression, the upper punch resumes its path on the cam track to its upper position as the rotary table 244 moves the punch out of alignment with the roller 254 in annular rotary direction 258 (See also FIG. 31).

In a similar context, the lower punch 256′ is in position aligned with lower roller 254′, which also is moved upward in the die at the same time the upper punch is moved downward The lower punches are normally located in position in the respective dies on the table so that the cavities of the dies are ready to be filled. The amount of powder filled into the die is controlled by the height of the lower punch as it moves beneath the powder feed frame 252. The lower punch height is adjusted by the weight adjustment cam. A conventional ejection cam 264, FIG. 30, (not shown in FIG. 31) ejects the tablet arrays from the associated die. It causes the lower punch to raise up and eject the tablet array from the associated die cavity.

In FIG. 31, the press is not in the same orientation as in FIG. 30 for simplicity of illustration. Also, in FIG. 31 the press 242 includes a second roller 260 which serves to precompress the particles forming the tablets. The circles 262 represent the formed tablet arrays and the positions of the corresponding punches. The ejected tablets 262 rest on the table 244 where positioned by the ejection process. The tablet arrays then impinge upon a separator 266 which causes the tablet arrays 262 on the rotating table 244 to be pushed in direction 270 onto an exit ramp 268 where they are collected by a collection device (not shown). The above description of the rotary press 242 is schematic only to show the general operation of the press, which is beyond the scope of the present invention. Still other known apparatuses (not shown) may be used to form the tablet arrays and to separate the arrays into individual tablets as discussed above. Such rotary presses may have 16 to 80 stations by way of example and may produce outputs in excess of 12,000 tablet arrays per minute.

As discussed above in the introductory portion, wet processes use no compression step as in a punch and die set and are not suitable for certain dry particles of interest which are moisture sensitive and can not be used to fabricate pellets in a wet process. These processes require water or some other solvent, which need to be avoided in certain implementations. The dry particles are for use with the apparatus and methodology to make tablets according to the various embodiments of the present invention.

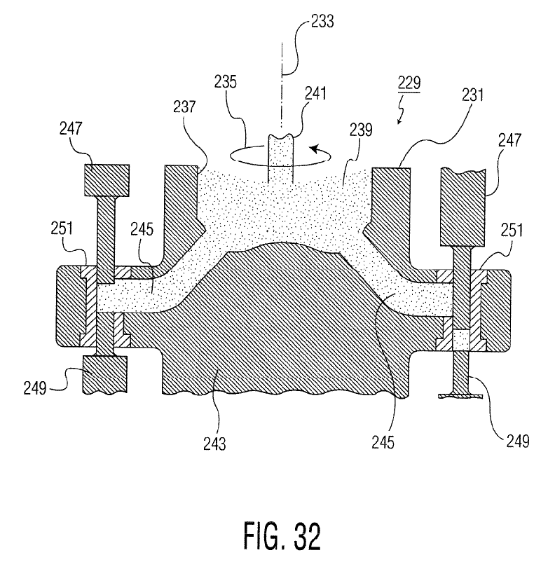

In FIG. 32, tablet forming apparatus 229 is of the centrifugal type discussed in the Introductory portion and known as the Comprima brand machine. This machine is available in four models 180, 230, 250 and 3000 having tablet forming speeds of 180,000, 230,00, 250,000 and 300,000 tablets per hour. Model 180 has 20 stations whereas model 300 has 36 stations with the other models having a number of stations between these values. Double tip tooling is available for doubling the production speed.

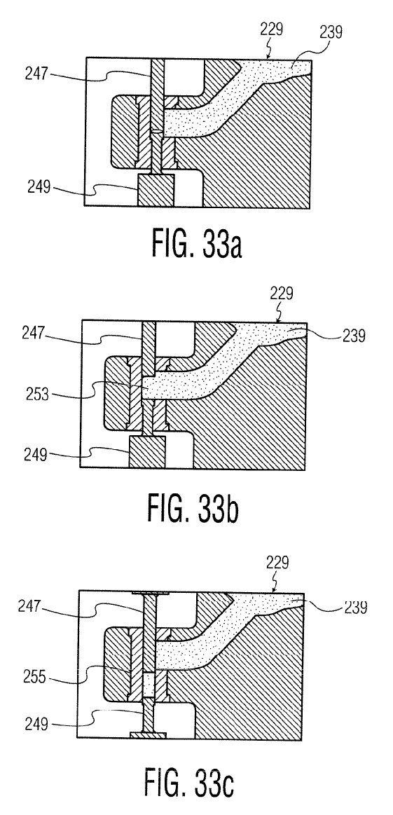

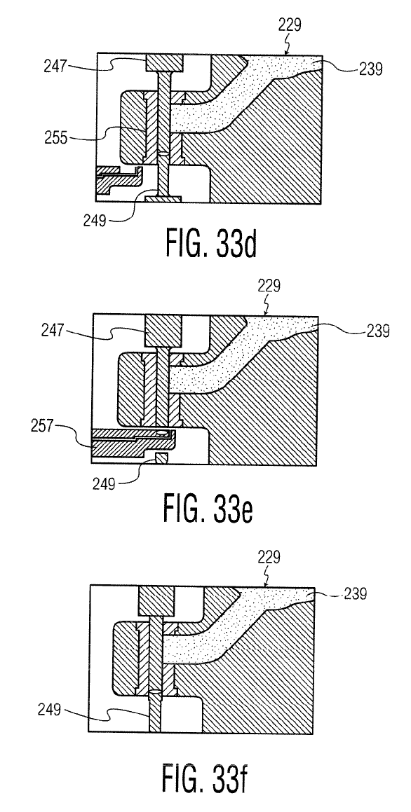

In FIG. 32, apparatus 229 comprises a rotary housing 243 containing a hopper 231 that rotates about axis 233 in the direction of the arrow 235. The hopper 231 has a cavity 237 that is filled with medicament powder 239 via a feed conduit 241. The housing 243 has an annular array of radially and laterally outwardly extending punch feed passages 245. Upper punches 247 and lower punches 249 are disposed in vertically oriented cavities 251 in the housing 243. The tips of the punches 247 and 249 are configured according to the present invention as described above in accordance with the various embodiments for fabricating tablets as described. The cavities 251 in the region between the upper punch 247 and lower punch 249 may be configured as shown by the various dies illustrated in the various embodiments above such as for example as shown in FIG. 15 and as shown schematically in FIG. 33.

In operation of the apparatus 229 of FIG. 32, the sequence of involved steps a-f are illustrated in FIG. 33. In FIG. 33, In step a, the upper and lower punches 247, 249 in contact with one another. When the punches separate, step b, the upper punch moves upwardly so that a sampling volume 253 is created between the two punches. The powder 239 flows into this volume 253 as result of centrifugal force of the rotating apparatus 229 and the vacuum created by the separation of the upper ane lower dies. The two punches then move downward together in unison, step c, lowering the sampling volume of powder with them. This brings the sampling volume of powder 253 into the closed lower part of the die 255. The upper punch is then lowered in the compression cycle, step d. This results in compaction, pre-compression and final compression. In step e, mechanism 257 ejects the formed tablets from between the upper and lower punches. The two punches are then raised and returned in contact for recovering the initial position of the punches, step e and the cycle repeats. As a consequence, the die 255 is filled by centrifugal forces due to rotation of the die table of apparatus 229 and vacuum force caused by the rapid separation of the punches during the filling step. That is the rapid separation of the punches creates a vacuum therebetween which draws the powder into the die volume between the two punches.

When the punches are in the so called contact phase, steps a and e, there may be a narrow space of 0.5 mm between the mating faces of the upper and lower punches.

Tests were run on the apparatus of FIGS. 32 and 33 with punches for creating tablets of 1 mm and 1.3 mm diameter. The 1 mm punches kept breaking. The 1.3 mm tools bent once, but a machine adjustment allowed them to run for about 4-6 hours without breaking. It is assumed they would keep on running without breaking. By making interconnected tablets of 1 mm or smaller diameter, the punch tips are strengthened and it is believed that they can thus be run without breaking.

It will occur to those of ordinary skill that various modifications may be made of the disclosed embodiments. For example, a tray may be provided in place of a lower punch and the die. The tray would serve both as a die and a lower punch. The upper punch may be used to provide the necessary compression forces. Such a tray would be similar to an ice cube tray, but would be robust for withstanding high compression forces. The upper die would have the configuration of the final tablet array where as the tray provides the cavities which are relatively open and readily suitable to receive particles of various dimensions. The punch defines the individual tablets and interconnecting links or bridges such as in FIG. 27 in cooperation with the mating die such as die 200, FIG. 24.

The punch creates the tablet array from the particles stored in a common cavity. In this manner micro sized tablets may be readily formed regardless the size of the particles to the size of the tablets. This overcomes a major problem with present punch and die sets forming individual tablets rather than arrays with weakened interconnecting links as described.

There thus has been shown apparatus and methodology for making a tablet from a configuration comprising an array of interconnected tablets and an array of links each forming an interconnection between each adjacent pairs of tablets, and which interconnections form weakened regions. It should be understood that the term “link” as used herein is intended to mean a junction between adjacent tablets, however long or short that junction may be and further, refers to a weakened region between adjacent tablets. The weakened region permits the tablets to be readily separated from each other.

For example, in FIGS. 10a, 10b, and 10c, the tangential regions between adjacent tablets form a link as defined herein. The array of FIG. 6 also forms links between adjacent tangential contiguous tablets of the array. Thus term link as used herein is not intended to be limited to separately distinguishable links of finite lengths such as links 96 of FIG. 8, links 92 of FIG. 12 or links 130 of FIG. 9, for example. Such links in practice may be shorter in length than the diameter of the corresponding tablets and thus may be less than 1 mm in length, thickness and width for 1.5 mm diameter tablets. Contiguous tablets also form such links as defined herein. The term tablet is defined as any structure formed by compression and of dry particles with no moisture involved. Such tablets may be used by sprinkling directly onto food or may be used in digestible known capsules, such as hard gelatin capsules, according to a given implementation.

The interconnections disclosed herein which form multiple tablets should be distinguished from conventional tables which are formed with weakening grooves. Such tables are single tables, riot separately defined tablets with interconnections as defined herein. A tablet with a weakening groove may be broken into separate subtablets whose dosage may be variable depending upon how the tablet is broken. such broken tablets would not be marketed as separate tablets as would the tablets of the present invention. The interconnections of the present invention provide predefined tablets.

It should be understood that the tablets of about 1.5 mm or less diameter are typically utilized as an intermediate product in the formation of capsules or other dosage forms into which the tablets are inserted or utilized. In the alternative, the tablets may be later further processed by adding a coating to control the release of the active ingredient from the tablet. The coated tablets are then inserted into a capsule or formed into another dosage form.

There also has been shown and described herein a punch for producing tablets comprising a shank and a punch tip attached to the shank. The punch tip has a base and at least one stanchion extending from the base. The at least one stanchion for defining an array of tablets. The tablets each have a body and an array of links interconnecting the array of bodies. There also has been shown a die for use with a pair of punches as described above.

The die cooperates with the pair of punches. The die has a single cavity for producing an array of interconnected tablets in cooperation with the pair of punches

There also has been shown a tablet forming punch and die set which form an array of tablets. The set comprises an upper tablet punch, a lower tablet punch, and a die having a cavity cooperatively receiving the upper and lower punches. The die and punches are arranged to form a tablet of given transverse and height dimensions. The punch and die are further arranged to configure the cavity to receive pharmaceutical powder particles that form the tablet. The arching index of the powders may be greater in at least one dimension than the given transverse and height dimensions. The various embodiments are given by way of illustration and not limitation. It is intended that the invention be defined by the appended claims.

What is claimed is:

1. A tablet configuration comprising:
an array of interconnected tablets; and
an array of links each forming an interconnection between each adjacent pairs of tablets, and which interconnections form weakened regions.

2. The array of claim 1 wherein the plurality of tablets have a given composition, the links having the same composition as the tablets.

3. The array of claim 1 wherein the tablets generally are substantially similar shaped discs.

4. The array of claim 1 wherein the tablets are generally spherical.

5. The array of claim 1 wherein the tablets are generally in the shape of a pyramid.

6. The array of claim 5 wherein the tablets are in the shape of mirror image pyramids.

7. The array of claim 1 wherein the tablets arc generally square in plan view.

8. The array of claim 6 wherein the tablets are rectangular in side elevation view.

9. The array of claim 1 wherein the tablets have a first height h and the links have a thickness t, the height h being greater than the thickness t.

10. The array of claim 1 wherein the tablets have a first height h and the links have a thickness t, the height h being the same as the thickness t.

11. The array of claim 1 wherein the links are dimensioned to form the weakened regions sufficiently weak so that the tablets separate from each other in the presence of an applied force or forces.

12. The array of claim 11 wherein the applied force or forces are induced by vibrating the tablet array.

13. The array of claim 1 wherein the tablets are circular and are interconnected to each other in tangential regions, the tangential regions forming said links.

14. The array of claim 1 wherein the links and tablets have a respective width dimension w_{L} and w_{T}, the width dimension w_{L} of the link being less than that of the tablet w_{T}.

15. The array of claim 1 wherein the links have a length dimension defined by the spacing between the tablets of the array of 0 mm to a value greater than 0 mm.

16. The array of claim 1 wherein the links each have a transverse cross sectional area less than any transverse cross sectional area of any of the tablets.

17. The array of claim 1 wherein the tablets are identical.

18. A punch for producing tablets in cooperation with a die comprising:
a shank; and
a punch tip attached to the shank, the punch tip having a base and at least one stanchion extending from the base, the at least one stanchion for defining an array of cavities, each cavity corresponding to a tablet having a body formed with the cavity and an array of cavity interconnecting passages for forming links interconnecting the array of bodies.

19. A die for use with a pair of punches according to claim 18 and for further defining said array of cavities with said at least one stanchion.

20. A die for cooperating with a pair of punches, each punch according to claim 18, the die having a single cavity further defining the array of interconnected tablets in cooperation with said at least one stanchion of said pair of punches.

21. The punch of claim 19 wherein the at least one stanchion comprises a plurality of stanchions that define a tablet body between adjacent stanchions and/or between adjacent stanchions and the die and having a transverse width w of no more than about 1.5 mm.

22. The punch of claim 18 wherein the at least one stanchion and base cooperate to define a circular cylindrical tablet body.

23. The punch of claim 18 wherein the at least one stanchion and base cooperate to define a portion of a generally square tablet body.

24. The punch of claim 18 wherein the at least one stanchion and base cooperate to define a generally pyramidal tablet body.

25. The punch of claim 18 wherein the at least one stanchion and base cooperate to define a portion of a generally spherical tablet body.

26. The punch of claim 18 wherein the at least one stanchion and base cooperate to define a portion of a generally rectangular tablet body.

27. A tablet made with the punch of claim 18.

28. A tablet forming punch and die set for forming an array of tablets comprising:
an upper tablet punch;
a lower tablet punch; and
a die having cavity cooperatively receiving the upper and lower punches wherein the die and punches are arranged to form a tablet of given transverse and height dimensions, the punch and die being further arranged to configure the cavity to receive at least one particle forming the tablet, the at least one particle having at least one dimension or an arching index greater than the given transverse and height dimensions.

29. The punch and die set of claim 28 wherein the cavity of the die is arranged to form a plurality of said tablets.

30. The punch and die set of claim 28 wherein the punches are arranged to form the plurality of tablets as an array of interconnected tablets.

31. A tablet made with the punch and die set of claim 28.

32. A method of forming tablets comprising forming an array of interconnected tablets having an array of separate and discrete weakened regions between adjacent tablets.

33. The method of claim 32 including forming the array into a plurality of separate discrete tablets by breaking the array apart at each of the weakened regions.

34. The method of claim 32 including applying a force to the weakened regions of the array to separate the tablets into discrete individual tablets.

35. The method of claim 32 wherein the step of forming the tablet comprises forming a cavity sufficiently large to form said array, filling the cavity with particles forming the array regardless the shape, size and arching index of the particles, and then compressing the particles to form said array.

36. The method of claim 32 including forming a cavity larger than any dimension of any tablet of the array, filling the cavity with particles forming the tablets, and then compressing the filled cavity to form the array.

37. The method of claim 32 including the step of breaking the array at each interconnection of the tablets to form the array into separate discrete tablets

38. The method of claim 32 including the step of removing the interconnections from each tablet to form the interconnected array into a plurality of discrete separate tablets.

39. The method of claim 38 including the step of forming each of the tablets in the range of up to about 1.5 to 2.0 mm in any dimension across the tablet.

40. A tablet made by the method of claim 34.

41. A tablet made by the method of claim 37.

42. A tablet made by the method of claim 38.

43. Tablet forming apparatus comprising:
a rotatable hopper for receiving powdered medicament for forming tablets,
a plurality of tablet forming stations arranged in an array about the hopper;
a plurality of powder feeding conduits coupled to the hopper for feeding the powdered medicament to the stations by centrifugal force; and
a punch and die set at each station for receiving the fed powdered medicament and for forming tablets from the medicament;
at least one of the punch and die sets at at least one of the stations is for producing an array of interconnected tablets;
wherein the interconnected tablets comprise an array of links each forming an interconnection between each adjacent pairs of tablets, and which interconnections form weakened regions which upon severance form separate and distinct tablets.
